(19) United States
(12) Patent Application Publication
(10) Pub. No.: US 2003/0027318 A1
(43) Pub. Date: Feb. 6, 2003

(54) Cytostatic factor

(75) Inventors: Avi Treves, Bar-Kochva (IL); Vivian Barak, Hertzel (IL)

Correspondence Address: Thomas C. Gallagher, ImClone Systems Incorporated, 7th Floor, 180 Varick Street, New York, NY, 10014, US

(73) Assignee: Hadasit Medical Research Services and Development, Ltd.

(21) Appl. No.: 09/863,659

(22) Filed: May 21, 2001

Related U.S. Application Data
(63) Continuation of application No. 08/779,189, filed on Jan. 6, 1997, now abandoned.
Application No. 08/779,189 is a continuation-in-part of application No. 07/915,144, filed on Jul. 17, 1992, now abandoned.
Application No. 07/915,144 is a continuation-in-part of application No. 07/766,433, filed on Sep. 26, 1991, now abandoned.
Application No. 07/766,433 is a continuation-in-part of application No. 07/525,274, filed on May 17, 1990, now abandoned.
Application No. 07/525,274 is a continuation-in-part of application No. 07/508,999, filed on Apr. 12, 1990, now abandoned.
Application No. 07/508,999 is a continuation of application No. 07/354,330, filed on May 19, 1989, now abandoned.

(51) Int. Cl. C12N 9/64; C07H 21/04; C12P 21/02; C12N 5/06

(52) U.S. Cl. 435/226; 435/69.1; 435/320.1; 435/325; 536/23.2

(57) ABSTRACT

The present invention is directed to a substantially pure protein that has cytostatic but not cytotoxic activity. The invention also provides a continuous cell line that produces the protein, and methods for its purification. The invention is also directed towards the use of the inhibitory protein, both in vitro and in vivo, in reducing the proliferation of bone marrow cells and leukocytes, modulating inflammation, as well as in immunosuppression and the treatment of cancer.

The present application is a continuation-in-part of application Ser. No. 07/915,144, filed Jul. 17, 1992, which is a continuation-in-part of application Ser. No. 07/766,433, filed Sep. 26, 1991, which is a continuation-in-part of Ser. No. 07/525,274, filed May 17, 1990, which in turn is a continuation-in-part of application Ser. No. 07/508,999, filed Apr. 12, 1990, which in turn is a continuation of application Ser. No. 07/354,330, filed May 19, 1989, each of which is incorporated by reference in its entirety.

### INTRODUCTION

The present invention is directed to a substantially pure protein that inhibits the proliferation of cells. The invention also provides a continuous cell line that produces the protein, and methods for its purification.

Vivian Barak et al., Eur. J. Immunol. (1986) 16:1449-1452, describe a cell line designated the M20 cell line that has not been deposited in any depository and is not available to the public. Barak et al. state that the M20 cell line produces what the authors concluded to be a specific IL-1 inhibitor. This conclusion was based on the authors' finding that crude supernatants and partially purified protein from M20 cells inhibit IL-1 dependent proliferation of cells.

### SUMMARY OF THE INVENTION

The subject invention provides a purified and isolated 52±8 kD molecular weight protein, free of the cell line M20 and M20-2, said protein being characterized by a pI of 4.1 and by having general cytostatic but not cytotoxic activity, including inhibition of IL-1 associated effects in vitro and in vivo.

The subject invention also provides a purified and isolated 52±4 kD molecular weight protein, free of the cell line M20 and M20-2, said protein being characterized by a pI of 4.1 and by having general cytostatic but not cytotoxic activity, including inhibition of IL-1 associated effects in vitro and in vivo.

### BRIEF DESCRIPTION OF THE FIGURES

FIG. 1: Fractions from a DEAE ion-exchange HPLC chromatography (0.0-0.3 M NaCI gradient) of supernatant from M20-2 cells were tested for IL-1 inhibition in the mouse thymocyte co-mitogenicity assay. Open circles represent the CPM of incorporated thymidine for triplicate cultures and closed circles represent protein concentration.

FIG. 2: Two active fractions from the comitogenicity assay of ion-exchange FPLC fractions were titrated to determine their change in inhibitory activity with decreasing concentration.

FIG. 3: Fractions from an ion-exchange chromatography were subjected to gel-fitration HPLC. The resulting fractions were tested for inhibition in the co-mitogenicity assay, and the peak of inhibitory activity was indicated at a molecular weight of approximately 50 kD. Cross hatching: peak of inhibitory activity.

FIG. 4: IL-1 and various amounts of the cytostatic factor were injected concomitantly into mice, and the percent-inhibition of fever was determined at different time points.

FIG. 5: IL-1 was injected into mice from 0 to 24 hours after the cytostatic factor and the percent-inhibition of fever was determined at two different time points. Cross-hatching to the upper-right: inhibition 1.5 hours after IL-1 injection. Cross-hatching to the upper-left: inhibition 1 hour after IL-1 injection.

FIG. 6: 150 units/mouse of IL-1, with and without the cytostatic factor, were injected into mice, and the percent inhibition of leukocytosis induction was determined. Each represents the average determination of the index of leukocytosis, which is the difference between time 0 leukocyte count and three hours post injection of five mice.

FIG. 7: Various sources and purities of cytostatic factor were fractionated on superose 12 FPLC and tested in the thymocyte proliferation assay. Molecular weight was determined using Bio-Rad standards. The results are shown in triplicate ± SEM.

FIG. 8: The pH gradient and inhibitory activity of the 20 fractions isolated from the Rotofor cell, in which the crude conditioned medium was fractionated, in the presence of carrier ampholytes (Bio-lyte) covering the pH range of 3-5.

FIG. 9: Profile of protein concentration and inhibitory activity of fractions eluted from the Sephacryl S-300 gel filtration column. The peak indicated between the two vertical lines was taken for the fractionation on the Rotofor cell.

FIG. 10: The pH gradient and distribution of protein concentrations (10A) and the inhibitory activity (10B) of the 20 fractions obtained from the Rotofor cell, the active peak eluted from the Sephacryl 3-300 gel filtration column.

FIG. 11: Analysis by PAGIF (pH 3-10) of the crude conditioned medium and some of the fractions collected from the Rotofor cell, when Affi-Gel blue was used as a preliminary step. Lane 1 and 5 pI markers (low pI calibration kit, Pharmacia); lane 2 and 3 crude conditioned medium; lane 4 fetuin 10 μg; lane 6 fraction pI 7.4; lane 7 fraction pI 6.07; lane 8 fraction pI 5.40; lane 9 pI 5.10; lane 10 fraction pI 4.66; lane 11 fraction pI 4.35; lane 12 fraction pI 4.12.

FIG. 12: Analysis by PAGIF (pH 3-6) of the most active fraction (fraction No. 10) from the Rotofor cell, in which gel filtration was used as a preliminary step. Lane 1 BSA 10 μg; lane 2 fetuin 10 μg; lane 3 cytostatic factor; lane 4 recombinant IL-1ra 5 μg; lane 5 pI markers.

FIG. 13: Analysis by SDS-PAGE of cytostatic factor purified by the sequence of purification procedures, as described. Lanes 1 and 4 MW markers (BSA, carbonic anhydrase and cytochrome c); lanes 2 and 3 the purified cytostatic factor. Bands may represent purified protein (cytostatic factor) or may represent co-purified protein that co-migrates with the cytostatic factor.

### DESCRIPTION OF THE INVENTION

The claimed protein is secreted by a myelomonocytic cell line, designated M20-2, that has been deposited in the American Type Culture Collection. The protein is a cytostatic factor that exhibits a number of clinically useful properties not previously described, such as general cell proliferation inhibition and lack of cytotoxicity. As would be expected of a protein, the inhibitory activity is destroyed by proteases, organic solvents, 2 mercaptoethanol, SDS, and extremes in pH and temperature. The term “inhibitory activity” as used in this specification includes cytostatic but not cytotoxic activity. Upon removing the factor, the inhibition is reversed.

The protein in question has a molecular weight that is difficult to determine precisely because of the low quantity isolated from the M20-2 cells. A molecular weight of 52±8, alternatively 52±4, was determined by open column gel filtration. A molecular weight of 55±7, alternatively 55±4, was determined by FPLC Superose 12 (Pharmacia) gel filtration. Sufficient quantities of the protein exhibit single bands on SDS-PAGE in the absence of 2-mercaptoethanol within the above molecular weight ranges. The protein has an isoelectric point of 4.1±0.2, and is obtainable by ion-exchange chromatography and HPLC purification from the supernatant of M20-2 cell line.

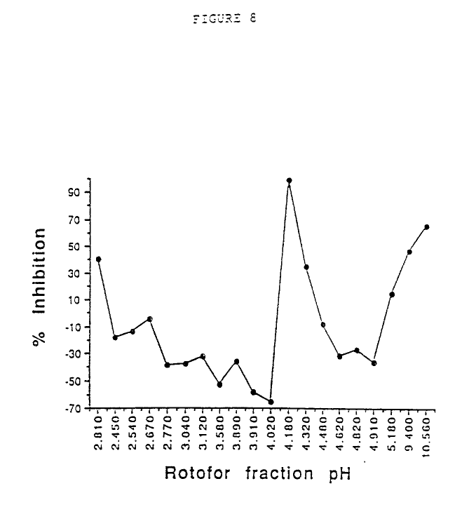

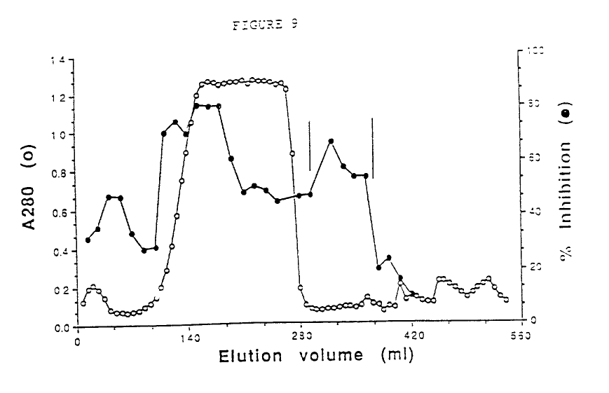

In one suitable purification procedure, preliminary fractionation of the conditioned medium from M20-2 cell cultures by isoelectric focusing (IEF) in the pH range 3-10 revealed a peak of inhibitory activity at approximately pH 4.0. Subsequent runs were all conducted in the narrower pH range 3-5. When the crude conditioned medium was applied in a Rotofor cell without a preliminary fractionation step, the sample was first concentrated by vacuum ultrafiltration or lyophilization and dialyzed against distilled water to reduce the concentration of salts. Ten runs were performed under the conditions described and the results of a typical run are shown in FIG. 8. It is important to avoid isoelectric precipitation of the large quantity of contaminating proteins present in the conditioned medium, since the failure to do so might result in the clogging of the screen separators and prevent electrophoretic transport. Isoelectric precipitation may be avoided by means of gel filtration through a Sephacryl S-300 column. The elution profile of the proteins from this column is depicted in FIG. 9.

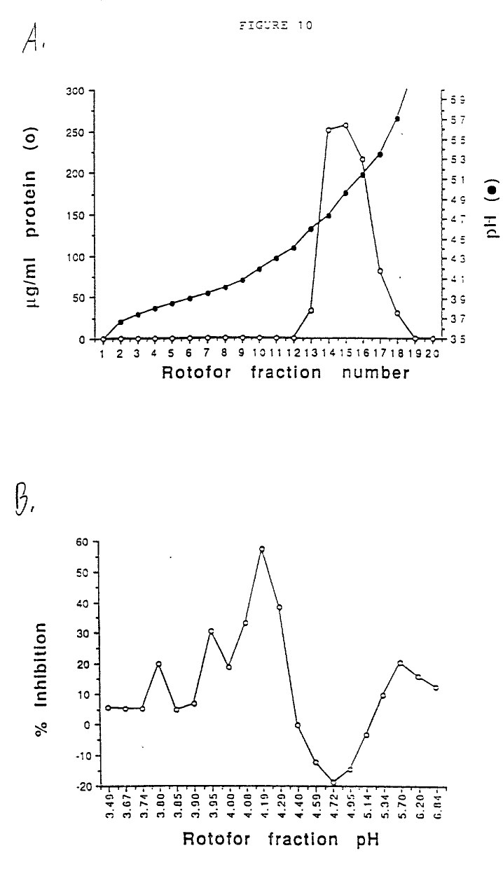

The inhibitory activity appeared under a broad area, and the small peak (Vₑ=524-595 ml) corresponding to a low protein content, as indicated by absorbance measurements (A_{280 nm}), was concentrated and dialyzed before fractionation by isoelectric focusing. The pH gradient, protein concentration and inhibitory activity of the 20 fractions isolated in the horizontal Rotofor column are shown in FIGS. 10A and 10B. It can be seen from FIG. 10B that the inhibitory activity was localized in fractions 9-11, covering the pH range 4.08-4.29, with fraction 10 (pH 4.19) being the most active. About 38% of the inhibitory activity present in the fraction from the Sephacryl S-300 column, which was applied to the Rotofor cell, was recovered in these three fractions, while, as seen from FIG. 10A, the great majority of proteins was present in fractions 13 through 17 (pH range 4.59-5.34). In the bioassay, 1.875 μg protein of the fraction obtained from the gel filtration column gave 57.6% inhibition. The same degree of inhibition (57.3%) was obtained with 25 μl of fraction number 10 (pI 4.19) obtained in the Rotofor cell. Since the concentration of protein in this fraction is 1.5 μg/ml, it follows that the specific activity of the cytostatic factor in this fraction has increased by approximately 50 fold (Table V).

To separate the contaminating proteins with a similar pI but different size, FPLC with a Superose 12 column was used as a final step. The results obtained using the sequence of procedures given in Table V show a 1600 fold purification, with a recovery of 5.4% of the inhibitory activity. The apparent molecular weight (MW, app.) of the active cytostatic factor, purified as described, determined by FPLC using the Superose 12 column is 55±7 kD.

As an alternative method to avoid overloading the Rotofor cell, Affi-Gel blue columns were used as a preliminary step to remove BSA, which is one of the major proteins present in the crude conditioned medium. This was followed by IEF in the Rotofor cell (pH range 3-5) with refractionation in the narrower pH range 3.9-4.3, in which the cytostatic factor has focused in the first run. Following the refractionation step, the specific activity increased 4.38 fold but the recovery dropped drastically.

In addition to the inhibitory activity, the above methods are capable of detecting the presence of some factors that increased the thymocyte proliferation induced by IL-1 in the bioassay, as shown in FIG. 8. These factors, which focused at different pH values, might be heterogeneous degradation products of IL-1 or other cytokines produced by the M20-2 cell line.

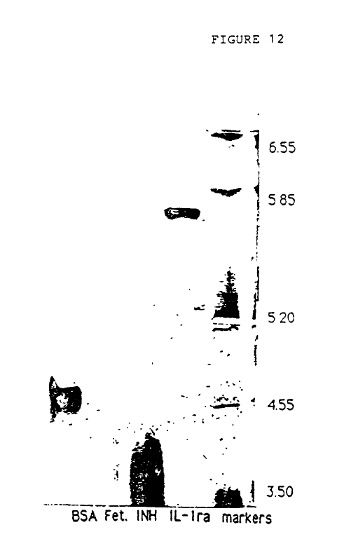

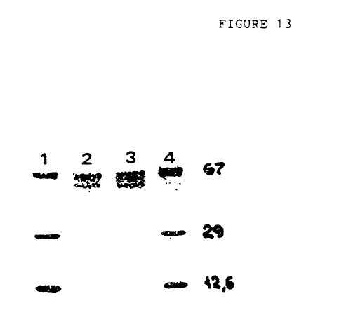

Aliquots of selected fractions obtained from the Rotofor cell after one run (pH 3-5), were concentrated 10-50 fold by ultrafiltration (ultracent-10 filters, Bio-Rad) and analyzed by PAGIF (FIG. 11). In the crude extract analyzed by PAGIF one can see that proteins with a pI smaller than that of BSA are present in minute concentrations. The cytostatic factor, which has a pI smaller than that of BSA as shown above, might therefore remain undetectable when the crude extract is analyzed by PAGIF. When the cytostatic factor separated from the bulk of protein present in the crude extract, and concentrated, was analyzed by PAGIF, it showed a few closely spaced protein bands of pIs smaller than that of BSA or the recombinant IL-1 receptor antagonist of the prior art (IL-1ra) (FIG. 12). By SDS-PAGE analysis, the cytostatic factor preparation obtained when Affi-Gel blue chromatography was used as a preliminary step shows the presence of two protein bands (FIG. 13). Similar results were obtained using gel filtration in the preliminary step. Amino acid sequencing revealed that the major protein band corresponds to fetuin derived from the fetal calf serum (FCS) used in the tissue culture medium.

To prove that the inhibitory activity is not due to fetuin or other FCS protein, medium supplemented with 10% FCS was fractionated by IEF at pH 3-5. None of the fractions corresponding to those generated from the conditioned media showed inhibitory activity. In addition, pure fetuin in relatively high concentrations did not have an inhibitory effect in the bioassay or in inhibiting IL-1 induced in vivo responses.

As outlined below, the claimed cytostatic factor protein has a high level of anti-inflammatory activity. Three art-recognized tests are typically employed to determine a given compound's ability to affect the inflammatory response, namely: effect on fever, leukocytes and enlargement of lymph glands. In each of these tests, the cytostatic factor had a significant effect in vivo in reducing each of these characteristic inflammatory responses. These results indicate a significant therapeutic potential for the novel cytostatic factor.

It is to be understood that the identity of the cytostatic factor is not so limited and encompasses various other amino acid substitutions, additions or deletions to the amino sequence of the described cytostatic factor.

Another embodiment of the present invention is recombinant production of the described cytostatic factor. Such process will employ DNA sequences that code for the cytostatic factor, and includes transforming with the recombinant DNA molecules, characterized by those sequences, various unicellular hosts to produce the cytostatic factor or portions thereof by fermentation of the transformed host. The present invention also employs the described cytostatic factor to determine the molecular structure and location of the active sites of the cytostatic factor and uses that information to design fragments and peptides for use as a cytostatic factor and for recombinant production as described in the uses and methods of the present invention.

The following is an outline of the techniques involved in the invention.

### Method of Purification

A number of methods may be used in purifying the subject cytostatic factor from supernatant of the M20-2 cell line. Briefly, a satisfactory level of purity, i.e., 1 unit of activity/3 μg of protein, has been obtained by ion-exchange chromatography, followed by further purification or ion exchange HPLC or FPLC. Alternately, ion exchange HPLC alone will provide a substantially pure product. In a preferred embodiment, the inhibitory protein is purified by isoelectric focusing. A brief description of each procedure is outlined below, and in greater detail in the examples in Section 6.

While the protocols noted above were used for each step of the purification, it is within the skill of the art to alter them slightly without significantly changing the results.

### M20-2 Supernatant Production

M202-2 cells were grown in RPMI supplmented with 10% FCS, penicillin (50 U/ml), streptomycin (50 μg/ml), MEM vitamin solution (1:100), 2 mM L-glutamine, 1 mM sodium pyruvate (Beit Haemek) (cRPMI=complete RPMI) at 37° C., 5% CO₂. After 4 days of growth, the cells were suspended in cRPMI without the addition of FCS at a concentration of 10⁶ cells/ml. These cells were incubated 24 hours under the same conditions. Supernatants were collected and concentrated by Pellicon (Millipore) or by vacuum ultrafiltration using dialysis tubing (cut-off 10,000±2000 kDa), lyophilized or dialyzed against a PEG slurry.

### Ion-Exchange Chromatography

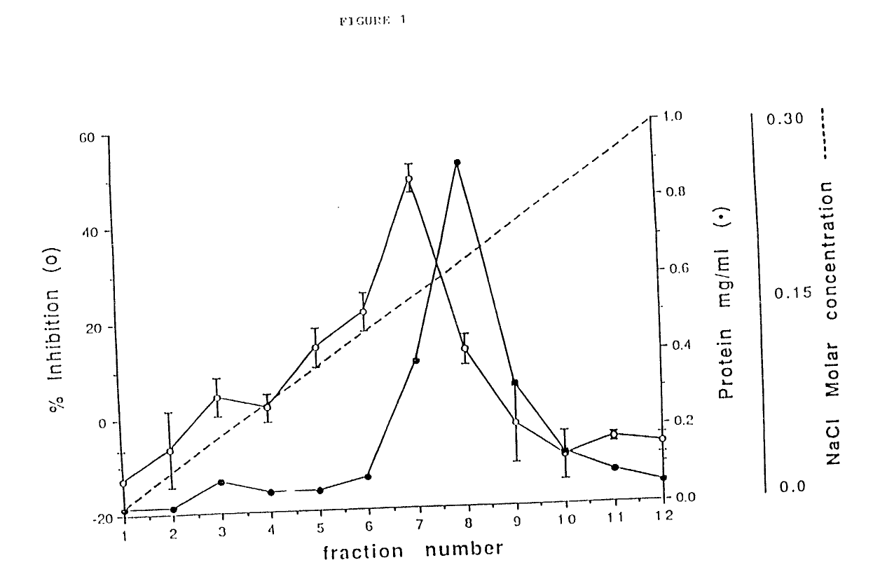

Supernatant from M20-2 cells, containing the cytostatic factor, was subjected to ion-exchange chromatography as noted above, and the resulting fractions were tested for inhibition in the co-mitogenicity assay. Fractions 11-15 (11-15), the peak of inhibitor activity (FIG. 1), were pooled, lyophilized and frozen for further characterization.

### Ion-Exchange HPLC

The pooled fractions, with peak inhibitory activity, from the ion-exchange chromatography were further purified by ion-exchange HPLC.

Fractions 11-15 from the ion-exchange chromatography were subjected to both HPLC and FPLC. ln the former, 300 mg of the lyophilized cytostatic factor was reconstituted in 2.0 ml of water and loaded onto a TSK DEAE-5PW HPLC column (Biorad) with a 0.0 to 0.1 M NaCl gradient in 20 mM Tris (pH 7.5) for 1 ml fractions per minute. FIG. 1 shows the inhibitory activity of the fractions, in pooled sets of two consecutive aliquots, as determined by a slightly modified co-mitogenicity assay. In the latter, 1.0 mg of the same cytostatic factor was reconstituted in 100 ml of water and loaded onto a Mono Q HR 5/5 FPLC column (Pharmacia) under the same conditions (except pH 8.0). The inhibitory activity of the fractions was determined as noted above (FIG. 1).

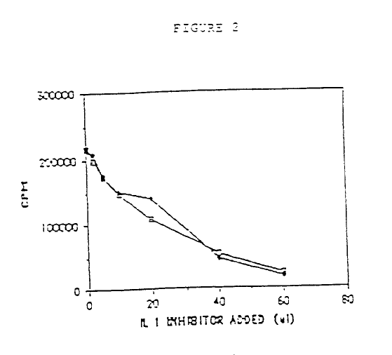

The inhibitory activity from the FPLC was titrated, using the co-mitogenicity assay under the conditions noted above (FIG. 2). Increasing amounts of two active fractions showed a similar increase in the inhibition of the assay.

### Gel-Filtration HPLC

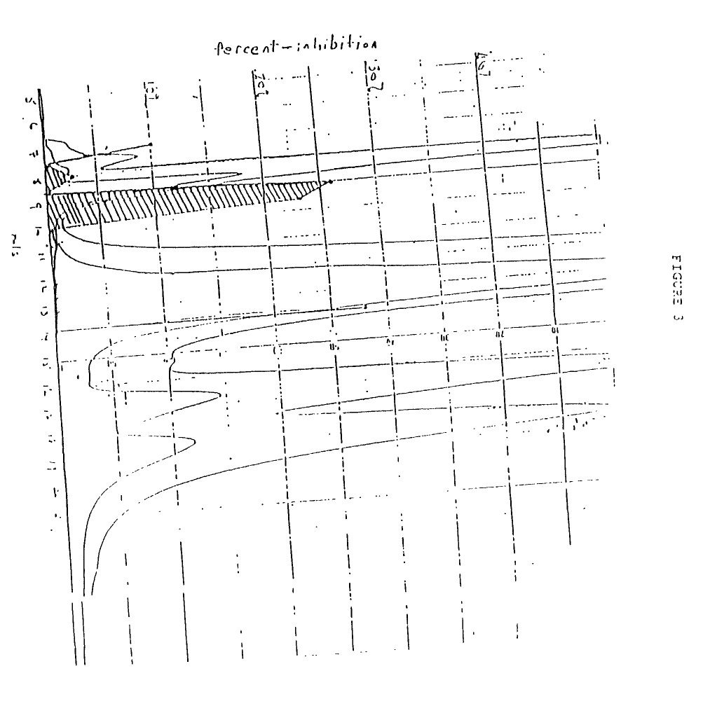

Lyophilized material from an ion-exchange chromatography was run on a Sorbax GF 250 HPLC column (Dupont). In FIG. 3, the peak of inhibitory activity (based on the co-mitogenicity assay noted above) is indicated at a molecular weight slightly less than that for the BSA standard, 68 kD, suggesting that in an unreduced state the cytostatic factor is approximately 50 kD.

### Isoelectric Focusing

Crude supernatant from M20-2 cells were also directly subjected to isoelectric focusing, without any prior purification. Of the fractions produced, the peak of inhibitory activity, as determined by the comitogenicity assay, was consistently found in the fraction that corresponds to a pI point of 4.1 to 4.2. The fraction was found to contain 300 units of activity, or 1 unit per 3 mg of protein. This fraction produced a single band on a non-denaturing (native), 10% gel stained with Coomassie blue. When slices of this gel corresponding to a range of molecular weights were sectioned and individually tested in the co-mitogenicity assay, the band produced by the pI 4.1-4.2 fraction corresponded exactly with inhibitory activity. There was no inhibitory activity in any of the other slices of the gel corresponding to other molecular weights, except for the very low molecular weight area at the bottom of the gel. Because activity was found in this region even for control runs that did not contain any fractions from the isoelectric focusing, it was not due to degradation of the cytostatic factor. This phenomenon is possibly due to break-down products of the gel itself. The band that is visualized with Coomassie blue and that corresponds to inhibitory activity is the fetal calf serum protein, fetuin. Fetuin does not in itself cause inhibitory activity in the thymocyte proliferation assay at the concentrations extracted from the gels. Nor does fetuin have any of the in vivo effects of the cytostatic factor described in this specification. Moreover, when protein secreted from the M20-2 cells are radioactively labeled (³⁵S-methionine), the biologically active fraction containing primarily fetuin contains a peak of radioactively labeled protein.

### Biochemical Characterization

The subject cytostatic factor has been shown to be effective in co-mitogenicity assays of inhibiting the co-mitogenic effect of IL-1 on mouse thymocytes. Significant inhibition of cell growth is seen after two to three days.

In addition, this factor has been seen to inhibit other cell lines, both cytokine dependent and independent. This inhibition does not correspond with cell death and therefore the factor is cytostatic but not cytotoxic. The cytostatic factor inhibits proliferation of all types of cells. For example, the cells may be eukaryotic, such as mammalian cells, including hematopoietic cells. The cells may also be prokaryotic cells, such as bacterial cells.

Table I below lists cells tested in vitro with the cytostatic factor. All cells were tested by incubating semi-purified cytostatic factor for three or more days with the cells at concentrations of 11-33% v/v.

TABLE I
EFFECT OF THE CYTOSTATIC FACTOR ON TRITIATED
THYMIDINE UPTAKE AND CELL COUNT
AFTER 3 DAYS ON VARIOUS CELL LINES

| | | | cell | |
|---|---|---|---|---|
| cell line | description | adher/suspen | count^{a} | cpm^{b} |
| NC | normal human glial cell | adherent | −/+ | − |
| G18 | human glialblastoma | adherent | ++ | − |
| A549 | human non-small cell carcinoma | adherent | ++/+ | − |
| Fetal | T antigen immortalized normal | adherent | + | + |
| spleen | fetal spleen | | | |
| B16.F10 | murine melanoma | suspension | +++ | ++ |
| HS | human plasmacytoma | suspension | ++/+++ | −/+ |
| HEL 92.17 | human erythroid leukemia | suspension | + | −/+ |
| Daudi | human Burkitt's lymphoma | suspension | died | died |
| NS1 | murine myeloma | suspension | +++ | +++ |
| B9 | murine B cell line (IL6 | suspension | +++ | +++ |
| | dependent) | | | |
| CTL-L | murine cytotoxic T lymphocyte | suspension | ++ | +++ |
| | (IL2 dependent) | | | |
| thymocytes | normal murine thymocytes | suspension | ND | +++ |
| | (mainly T) | | | |
| SKW 6.4 | human transformed B cell | suspension | ND | +++ |
| HL-60 | human myeloid leukemia | suspension | +++ | +/++ |
| M20 | human myeloid leukemia | suspension | +++ | +++ |
| U937 | human myeloid leukemia | suspension | ND | ++ |
| K562 | human myeloid leukemia | suspension | ND | + |
| MEL | murine | suspension | ND | ++ |
| WEHI | murine | suspension | +++ | + |
| | human fresh PB myeloid leukemic | suspension | ND | +++ |
| | cells | | | |

| | normal human fresh non-adherent PB (treated | ND | +++ |
|---|---|---|---|
| | W/GM-CSF) | | |
| ^{a}Cells were extracted from wells and on day 3 were counted in the presence of trypan blue using a hemocytometer. No significant cytoxicity was observed. A “−” indicates no difference in cell count between cells treated with inhibitor and control cells. A “+” indicates a 10-30% decrease in cell number in inhibitor wells. A “++” indicates a 30-50% decrease in cell number in inhibitor wells. | | | |
| #A “+++” indicates an over 50% decrease in cell number in inhibitor wells. | | | |
| ^{b}Tritiated thymidine was added for the last 8-18 hours of a 3 day growth and harvested onto glass fiber strips. A “−” indicates no difference in CPM between cells treated with inhibitor and control cells. A “+” indicates a 10-30% decrease in CPM in inhibitor wells. A “++” indicates a 30-50% decrease in CPM in inhibitor wells. A “+++” indicates an over 50% decrease in CPM | | | |
| #in inhibitor wells. | | | |

### Activity In Vivo

For all of the in vivo assays, recombinant human IL-1 (Cistron) and recombinant mouse IL-1 were dissolved in PBS (pH 7.2) and stock solutions of 10 units/ml were stored at −20° C. “One unit of activity” for IL-1 was defined as the amount required to augment by 50% the response of mouse thymocytes to PHA in the comitogenicity assay. In addition, cytostatic inhibitor purified by ion-exchange chromatography (gradient 0.0 to 0.4 M NaCl) was used in all of the in vivo assays. Fractions were dialyzed against RPMI 1640 and stored at −20° C., with “one unit of activity” for the cytostatic factor defined as the amount required to inhibit by 50% the co-mitogenic activity of one unit of IL-1. For both IL-1 and its cytostatic factor, as well as Con A and LPS, titration of activity was performed at twofold dilutions in order to determine the number of units used. The percentage of inhibition was calculated as:

$$100 \times \frac{[ 1 - ( ACTIVITY WITH CYTOSTATIC FACTOR ) ]}{[ ( ACTIVITY OF IL - 1 ALONE ) ]}$$

In all of the in vivo experiments, male Balb/c mice, 6 to 8 weeks old, were injected with PBS solutions of Con A, LPS or IL-1, alone or concomitantly with the cytostatic factor, and each experimental group usually consisted of four mice.

The experiments to assay the inhibition of fever induction were started only after there had been 3 consecutive and similar baseline temperature readings in each mouse. Rectal temperature was measured with a No. 402 Thermistor Probe (Yellow Springs Instruments, Yellow Springs, Ohio), inserted to a distance of 2 cm for about 30 seconds and recorded on a Yellow Springs Telethermometer. Mice were gently restrained during the procedure by their confinement in a size-compatible, ventilated tube. Experiments were performed in which either 0.25, 0.5, 1.0, 1.5 or 2.0 units of cytostatic factor and 150 units human IL-1 were intravenously injected concomitantly, and in which 2 units of cytostatic factor was intravenously injected from 2 to 24 hours before the 150 units of IL-1.

To measure the inhibition of leukocytosis induction, blood samples were taken from the mice by venous puncture of the tail and stored in heparinized tubes, with total leukocyte counts determined by a Coulter Model S-Plus (Coulter Electronics, Hialeah, Fla.). In one experiment, either 50, 100, 150 or 200 units of human IL-1 were intravenously injected either alone or concomitantly with 2 units of cytostatic factor; in another, 2 units of cytostatic factor were intravenously injected from 2 to 24 hours before 150 units of IL-1.

To assay the inhibition of lymph node-enlargement, 0.05 ml of the sample to be tested was injected subcutaneously into both right foot pads of a mouse to cause a response in the local draining lymph node, which was measured by an increase in lymph node weight. After 3 days, the mice were anesthetized with ether and sacrificed by cervical dislocation. The popliteal lymph nodes from both the right and left sides were removed, trimmed of fat and weighed. After previously determining the dose response and kinetics for optimal lymph node enlargement, those amounts, 12.5 μg Con A, 50 μg LPS, 125 units mouse IL-1 and 100 units human IL-1, were injected either individually or in combination with 2 units of cytostatic factor. Also, 1 unit of cytostatic factor was injected either from 2 hours to 4 days before 125 units of mouse IL-1 or from 2 hours to 1 day after the IL-1.

### Therapeutic Use of Cytostatic Factor

The cytostatic factor generally inhibits cell growth. In addition, the factor modulates inflammation and reduces various inflammatory parameters; reduces the proliferation of bone marrow cells and leukocytes but does not affect differentiation; and is useful in immunosuppression and the treatment of cancer.

For example, the data presented below show the utility of the subject cytostatic factor as an anti-inflammatory agent, presumably as a result of its inhibitory effect. Thus, the present invention also provides a method for prevention or treatment of an undesired inflammatory response. Among conditions which would involve undesirable inflammatory responses are allergic reactions, chronic inflammatory diseases, autoimmune diseases (e.g. rheumatoid arthritis) and high fevers associated with cancer or infectious conditions. The active component is preferably formulated into a composition in combination with a pharmaceutically acceptable carrier. Administration of the composition may be either oral or by local or parenteral injection. An effective unit dose form of the present composition would comprise approximately 0.0001-1.0 μg of the active component per kg of body weight. However, it is within the skill of the experienced physician to adjust the dose in accordance with the patient's needs and the condition to be treated. The cytostatic factor may also be used to inhibit proliferation of normal and leukemic mammalian myeloid progenitors (including human).

### Other Uses

The claimed cytostatic factor also has significant non-therapeutic uses as a tool in identification of the gene sequence. It is within the scope of the invention to produce large amounts of the cytostatic factor, substantially purified from other human proteins, using its DNA sequences, along with included DNA molecules in unicellular hosts transformed with the DNA sequence. The purified cytostatic factor can be used as a source of amino acid sequence data for use in designing DNA probes which can be used to isolate and select DNA sequences coding for the cytostatic factor of this invention.

In particular, the amino acid sequence of various sites and fragments of the purified cytostatic factor can be determined and used to deduce the DNA sequences coding for them to design DNA probes which are potentially useful to screen various DNA libraries for DNA sequences coding for the cytostatic factor. Sources of such libraries can include chromosomal gene banks and DNA or cDNA libraries of tissue or cell lines producing the cytostatic factor of the invention. Methods for preparing and cloning cDNA, screening libraries of clones, and expressing the protein in a suitable host are described in PCT International Application, Publication Number WO 89/01946 and in Maniatis et al., “Molecular Cloning: A Laboratory Manual,” Cold Spring Harbor Laboratory Press (1982).

Briefly, preparing cDNA involves isolating poly A⁺mRNA from an cytostatic factor producing cell source, followed by construction of a cDNA library from the isolated poly A⁺mRNA. The library of clones are then screened for the recombinant DNA molecule containing the cytostatic factor insert utilizing various approaches. One approach may include the use of DNA probes to screen cDNA or genomic libraries that encode for cytostatic factor. The DNA probes consist of a series of synthetic DNA fragments which are constructed based on partial amino acid sequences of the purified cytostatic factor determined by techniques well known in the art. The selected sequences may then be used to transform and express the cytostatic factor of the present invention in prokaryotic and eukaryotic hosts. The DNA sequences of the present invention may also be utilized in vectors consisting of segments of chromosomal, non-chromosomal and synthetic DNA and expressed in a variety of host/vector combinations to produce cytostatic factor. Derivatives of SV40 and known bacterial plasmids as expression vectors with various sites of inserted cytostatic factor DNA sequences and a control sequence, and other vectors capable of expression in prokaryotic and eukaryotic hosts can be employed. The present invention thus encompasses the present cytostatic factor or its equivalents, produced by any means, and is not merely restricted to a protein which has been isolated from the M20-2 cell line.

### EXAMPLE

### Purification and Activity of the Cytostatic Factor

The following is an example of the purification process, characterization and activity assays for the cytostatic factor from M20-2 cells.

### Method of Purification

At each stage of purification, cytostatic factor activity was measured by the inhibition of the IL-1 augmentation of the mitogenic response in mouse thymus cells to PHA, as described (Barak et al., Eur. J. Immunol. 16: 1449-1452, 1986). The inhibition of this IL-1 comitogenic activity was indicated by comparing the reduction in ³(H)-thymidine incorporation in the growing cells, in the presence of the cytostatic factor, with the incorporation in the growing cells in the presence of IL-1 alone. C3H/HeJ mouse thymocytes were cultured for 48 hours in 96 well plates, containing 1.0×10⁶ cells, 1 unit of recombinant human IL-1 (Cistron Biotechnoloqy, Pine Brook, N.J.) and 500 ng of phytohemagglutinin (PHA) in 150 μl of RPMI 1640 culture medium (with 10% fetal calf serum and 5×10⁻⁶M −β mercapto-ethanol) per well. The PHA mitogen concentration was previously determined by titration in the thymocyte assay. Cultures were pulse-labeled with 1.0 μC of 3(H)-thymidine before harvesting 24 hours later and data were based on counts per minute (CPM) of triplicate or quadruplicate cultures.

For the first step of purification by ion-exchange chromatography, 100 ml of supernatant from the cultured media of myelomonocyte cell line M20-2, a subline of M20 (Barak et al., Eur. J. Immunol. 16:1449-1452, 1986), was applied to a column (2.5×20 cm) of DEAE Sephacel (Pharmacia, Uppsala, Sweden), as described in Barak et al., id. The column was eluted with 50 mM Tris HCl buffer, pH 7.4 (200 ml), and then developed with a linear gradient of NaCl (0.0 to 0.6 M). Fractions were collected, dialyzed against RPMI 1640, filtrated and tested in 20, 50 and 100 μl aliquots per well for the inhibition of IL-1 activity in the co-mitogenicity assay of mouse thymocytes with PHA.

In the next step of purification by ion-exchange HPLC, initially lyophilized protein from the ion-exchange chromatography, after being run on a PD10 “desalting column” (G-10 column from Pharmacia), was reconstituted in water and run on an HPLC or FPLC column. From each resulting fraction, the inhibitory activity of 10 μl aliquots was determined in the co-mitogenicity assay by measuring the reduction of 3(H)-thymidine incorporation in the proliferating thymocytes.

As an alternative process for purifying the cytostatic factor, supernatant from the cultured media of M20-2 cells can be directly run on an ion-exchange HPLC column, without any prior purification.

In order to further characterize the cytostatic factor, lyophilized material from the ion-exchange chromatography was subjected to gel-filtration HPLC with a Sorbax GF 250 column (Dupont) Phosphate buffered saline (PBS, pH 7.4) was used as the buffer and 25 mg of the material was reconstituted in 250 μl of PBS, loaded as the sample and run at 0.5 ml per minute. From each 0.5 ml fraction, a 30 μl aliquot was analyzed for inhibitory activity, as before, in the co-mitogenicity assay.

In a preferred embodiment of the invention, the Rotofor cell (Bio-Rad, Richmond, Calif.) was used to carry out preparative isoelectric focusing; see Egen et al., Electrophoresis '83, H. Hirai ed., Walter de Gruyter & Co., Berlin, N.Y., pp. 547-550 (1984). Proteins were separated in this method by isoelectric focusing in free solution. The starting material for the isolation of cytostatic factor was 1 liter of crude supernatant obtained from the cell line M20-2. The supernatant was lyophilized and the powder obtained (15 g) was dissolved in RPMI 1640 and dialyzed against 1000 volumes of RPMI 1640 diluted 1:150. Prior to the run, the sample, containing 1.25-1.85 mg/ml of protein was diluted 20-fold with 10 M urea to have a final concentration of 5 M urea. Carrier ampholytes, Bio-Lyte 3/5, were added to obtain a final concentration of 2% (w/v). The volume of the mixture was 50 ml. As anolyte 0.1 M H₃P0₄, and as catholyte 0.1 M NaOH, were used. To bring the sample to the running temperature (4° C.), the cell was rotated for 15 minutes before applying electric current. The running conditions were typical as follows: constant power 12 watts; 466 volts and 26 milliamps. After 4 hours when the run was completed, the voltge reached 698 volts and the current was 17 milliamps. At the completion of the run, the pH gradient was determined on the 20 harvested fractions (2 ml each), diluted 1:10 with freshly boiled double-distilled water. For refractionation the fractions showing inhibitory activity, having a pH of 4.03 to 4.21, were pooled and diluted With 10 M urea to a final concentration of 5 M urea. The run was performed as above, without adding additional carrier ampholytes. The beginning running conditions were as follows: constant power 12 watts; 1087 volts and 12 milliamps. At the end of the run, the voltage reached 1828 volts and the current was 7 milliamps.

To remove the carrier ampholytes, the harvested fractions were dialyzed against 1 M NaCl, followed by dialysis against RPMI 1640. The purity of the fractions showing activity in the comitogenicity assay was determined by analytical isolectric focusing in 0.5 mm thin-layer polyacrylamide gels, using the LKB 2117 Multiphor System and by SDS-PAGE using the Mini-Protein 11 slab cell of Bio-Rad.

### Gel-Filtration Chromatography

Fractions of the cytostatic factor with peak inhibitory activity from an ion-exchange chromatography were analyzed by gel-filtration chromatography on a Sephadex G-200 Superfine column (Pharmacia). The samples were dialyzed, lyophilized, dissolved in a small volume of buffer and 2.0 ml was run. As before, each new fraction was assayed for its ability to inhibit the co-mitogenic effect of IL-1 on mouse thymocytes.

Peak fractions of inhibitory activity from both the ion-exchange and the gel-filtration chromatography (i.e. before and after the G-200 column) were run in adjacent lanes on a SDS-polyacrylimide electrophoretic gel with molecular weight markers.

### SDS-PAGE

The fractions of the cytostatic factor, from both the ion-exchange and the subsequent gel-filtration chromatography, were analyzed by SDS-PAGE (FIG. 13).

### Activity In Vivo

Cytostatic factor, from the peak of inhibitory activity from an ion-exchange chromatography of culture media from M20-2 cells, was shown to have anti-inflammatory activity by three art-recognized tests, i.e., effect on fever, leukocytosis and enlargement of lymph glands, as discussed below.

### Inhibition of Fever Induction

As described above, the cytostatic factor's ability to inhibit the induction of fever was tested in mice, with the results presented as “mean body temperature±standard error (SE)” for each group of two mice injected with the same compound. The differences in temperature within each group over time are given as ΔT values.

Body temperature was measured after increasing elapsed times (½ hour, 1 hour, 1 and ½ hours, and 2 hours) following the concomitant injection of 150 units of IL-1 and 1 or 2 units of the cytostatic factor, with control groups injected with PBS or human IL-1 alone. The results are shown in Table II.

TABLE II
INHIBITION OF IL-1 INDUCED FEVER BY CONCOMITANT INJECTION OF CYTOSTATIC FACTOR

| | | Amount | | | | | | | | | |
|---|---|---|---|---|---|---|---|---|---|---|---|
| | | of | | | | | | | | | |
| | | cyto- | | | | | | | | | |
| | | static | 0 | | 1 | | 1 1/2 | | 2 | | |
| | Inject | factor | Body | 1/2 | % | | % | | % | | % |
| Exp. | ion | per | temp. | ΔT* | Inh. | Δ T | Inh. | Δ T | Inh. | Δ T | Inh. |
| No. | PBS | mouse | ND | ND | ND | ND | ND | ND | ND | ND | ND |
| 1 | IL-1 | — | 37.7 | 0.22 ± 0.14 | — | 0.46 ± 0.14 | — | 0.24 ± 0.09 | — | 0.02 ± 0.11 | — |
| | plus | 2 units | 37.7 | −0.12 ± 0.12 | | −0.72 ± 0.18 | 100 | −0.22 ± 0.16 | 100 | −0.02 ± 0.11 | 100 |
| | cyto- | | | | | | | | | | |
| | static | | | | | | | | | | |
| | factor | | | | | | | | | | |
| 2 | PBS | — | 36.87 | 0.1 | — | 0.13 ± 0.09 | — | 0.13 ± 0.05 | — | 0.06 ± 0.07 | — |
| | IL-1 | — | 36.57 | 0.43 ± 0.04 | — | 1.3 ± 0.11 | — | 0.7 ± 0.10 | — | 0.22 ± 0.05 | — |
| | plus | 2 units | 36.72 | −0.2 ± 0.03 | 100 | 0.15 ± 0.05 | 100 | 0.08 ± 0.06 | 100 | 0.13 ± 0.06 | 100 |
| | cyto- | | | | | | | | | | |
| | static | | | | | | | | | | |
| | factor | | | | | | | | | | |
| 3 | PBS | — | 36.67 | 0 | — | −0.1 ± 0.06 | — | −0.05 ± 0.04 | — | 0± | — |
| | 1L-1 | — | 36.52 | 0.52 ± 0.02 | — | 1.52 ± 0.11 | — | 0.6 ± 0.06 | — | 0.22 ± 0.05 | — |
| | plus | 2 units | 36.67 | 0.13 ± 0.02 | 100 | −0.15 ± 0.06 | 95 | 0.03 ± 0.06 | 95 | 0.02 ± 0.06 | 89 |
| | cyto- | | | | | | | | | | |
| | static | | | | | | | | | | |
| | factor | | | | | | | | | | |
| 4 | PBS | — | 36.90 | 0.12 ± 0.04 | — | 0.12 ± 0.03 | — | 0.07 ± 0.02 | — | 0.07 ± 0.02 | — |
| | IL-1 | — | 36.70 | 0.45 ± 0.06 | — | 1.47 ± 0.09 | — | 0.7 ± 0.11 | — | 0.27 ± 0.1 | — |
| | plus | 1 unit | 36.47 | 0.2 ± 0.07 | 55 | 0.72 ± 0.08 | 50 | 0.4 ± 0.08 | 43 | 0.15 ± 0.02 | 45 |
| | cyto- | | | | | | | | | | |
| | static | | | | | | | | | | |
| | factor | | | | | | | | | | |

*ΔT: change in body temperature

In all four experiments a peak of increase in body temperature was observed 1 hour after injection with IL-1 alone, while a complete inhibition of temperature elevation was observed at all four time points, in all four experiments, when the cytostatic factor was concomitantly injected.

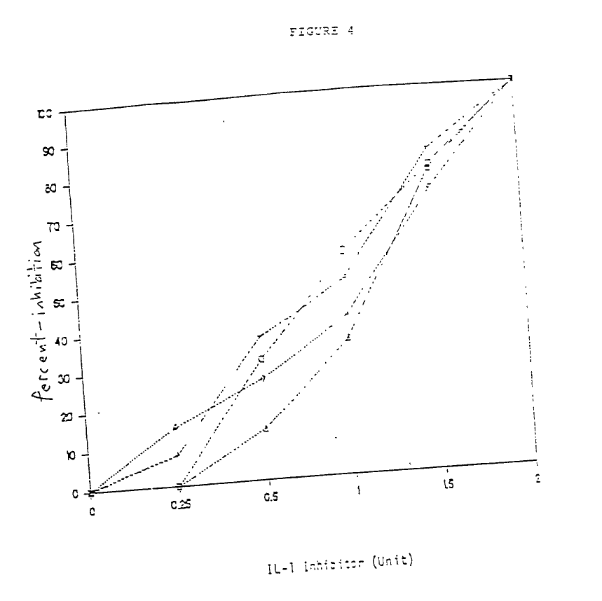

Next, the effect of varying amounts of the cytostatic factor on IL-1 fever induction was tested by measuring the body temperature at different time points after the concomitant injection of IL-1 and the cytostatic factor (FIG. 4). A gradual increase in the inhibition of fever with increasing amounts of cytostatic factor, independent of time, was observed, with a range of about 50% inhibition obtained with 1 unit of cytostatic factor.

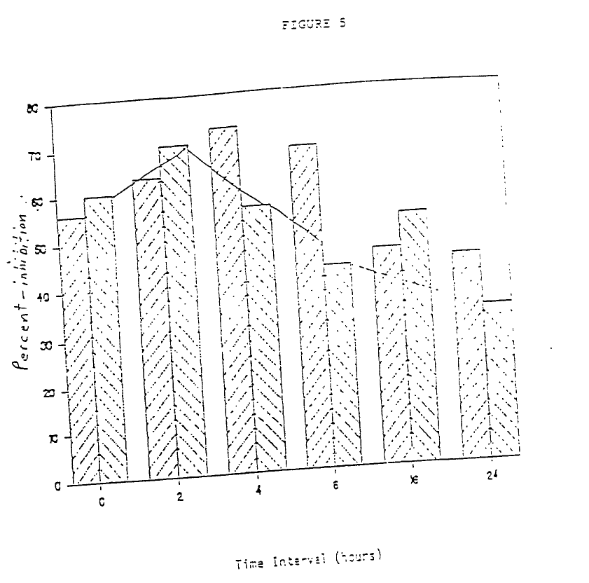

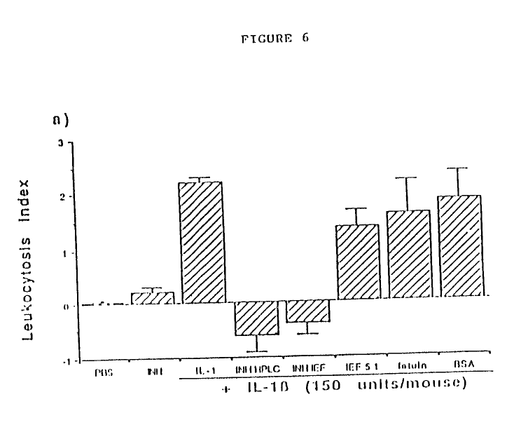

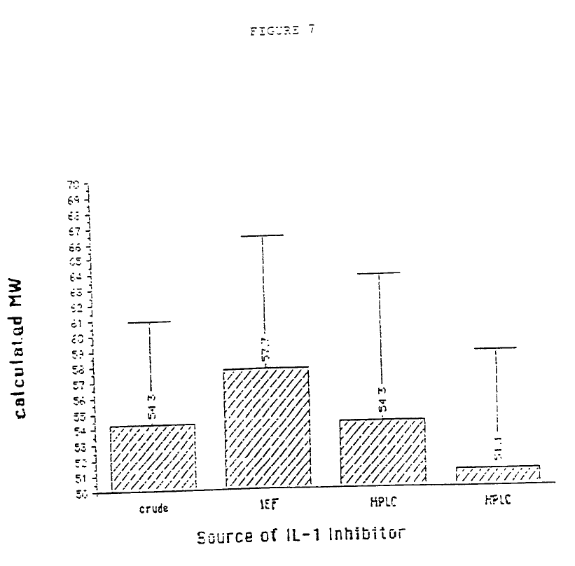

Finally, the effect of the time interval between the injection of 150 units of IL-1 and 2 units of the cytostatic factor on body temperature was tested at 1.0 and 1.5 hours after the IL-1 injection (FIG. 5). The results indicate that fever inhibition was exhibited when the cytostatic factor was injected from 2 to 16 hours before the injection of IL-1, with a decrease in inhibition when the cytostatic factor was injected up to 24 hours before. Thus, the cytostatic factor reversed the fever induced by IL-1 even if injected 24 hours before the IL-1.

### Inhibition of Leukocytosis Induction

The ability of the cytostatic factor to inhibit the induction by IL-1 of leukocytosis in mice was assayed by the index of leukocytosis, defined as the mean (±SE) leukocyte counts in a group injected with human IL-1 alone or in conjunction with the cytostatic factor, divided by the mean (±SE) counts in a group injected with PBS only (the standard error, for all data was between 0.0350 and 0.0127).

In general, the peak increase in leukocyte counts was observed 2 to 3 hours after the injection of 150 to 200 units of IL-1 per mouse. The concomitant injection of 2 units of cytostatic factor always caused a reduction of this IL-1 effect, with maximal Inhibition obtained 2 to 3 hours after the injection.

As found for fever induction, inhibition of leukocytosis was also observed when IL-1 and the cytostatic factor were injected separately, at varying time intervals. The cytostatic factor reduced the leukocytosis induced by IL-1 even if injected 2 to 24 hours before the IL-1, but the peak inhibitory effect, at 4 hours, was decreased if the time interval was increased.

### Inhibition of Local Lymph Node-Enlargement Induction

To assay the ability of the cytostatic factor to affect the induction, by various agents, of local lymph node enlargement, the index of enlargement was determined by the mean (±SE) weight of the right lymph nodes of the mouse (injected with IL-1 alone or in conjunction with the cytostatic factor) divided by the mean (±SE) weight of the left lymph nodes, injected with just PBS.

TABLE III
EFFECT OF CYTOSTATIC FACTOR ON LOCAL LYMPH NODE
ENLARGEMENT

| | | | Cyto- | | |
|---|---|---|---|---|---|
| | | | static | | |
| | Amount | | Factor | Index of | Percent |
| Inducer | Per Mouse | | (2 units) | Enlargement | Inhibition |
| Con A | 12.5 | μg | − | 3.75 ± 0.07 | — |
| | 12.5 | μg | + | 1.17 ± 0.04 | 69 |
| LPS | 50 | μg | − | 4.85 ± 0.27 | — |
| | 50 | μg | + | 1.19 ± 0.07 | 75 |
| Mouse | 125 | units | − | 4.09 ± 0.11 | — |
| IL-1 | 125 | units | + | 1.26 ± 0.11 | 70 |
| Human | 100 | units | − | 3.76 ± 0.06 | — |
| IL-1 | 100 | units | + | 0.89 ± 0.09 | 77 |

The results in Table III represent the optimal lymph node enlargement induced by mitogens such as Con A and LPS as well as both human and mouse IL-1. In all of these cases, the concomitant injection of 2 units of the cytostatic factor caused a significant reduction in the lymph node response.

The inhibitory effect on the induction by mouse IL-1 of lymph node enlargement was also tested when IL-1 and the cytostatic factor were injected separately, at varying time intervals. The results are shown in Table IV.

TABLE IV
EFFECT OF CYTOSTATIC FACTOR ON LOCAL LYMPH NODE
ENLARGEMENT

| Time Interval | | | | | |
|---|---|---|---|---|---|
| Between IL-1 and | Lymph Node | | | | |
| Cytostatic Factor | Weight (mg) | | Index of | | Percent |
| Injection | Right | Left | Enlargement | | Inhibition |
| IL-1 | 0.76 | 4.28 | 5.63) | 4.20 ± 0.42 | — |
| (no cytostatic | 1.12 | 3.94 | 3.51) | | |
| factor) | 0.94 | 3.82 | 4.06) | | |
| | 1.07 | 3.88 | 3.62) | | |
| 4 days before | 0.86 | 3.53 | 4.10) | 3.80 ± 0.21 | 10 |
| | 1.08 | 3.78 | 3.50) | | |
| 3 days before | 1.30 | 3.42 | 2.63) | 2.87 ± 0.17 | 31 |
| | 1.24 | 3.88 | 3.12) | | |
| 2 days before | 1.94 | 3.25 | 1.68) | 1.82 ± 0.1 | 56 |
| | 1.26 | 3.78 | 1.96) | | |
| 1 day before | 0.85 | 1.36 | 1.32) | 1.46 ± 0.1 | 65 |
| | 1.28 | 1.70 | 1.32) | | |
| 2 hours before | 0.96 | 1.56 | 1.62) | 2.23 ± 0.27 | 47 |
| | 1.42 | 3.98 | 2.80) | | |
| | 1.02 | 2.32 | 2.27 | | |
| 2 hours after | 1.05 | 2.96 | 2.81) | 2.24 ± 0.23 | 47 |
| | 1.38 | 2.56 | 1.85) | | |
| | 1.36 | 2.84 | 2.08) | | |
| 1 day after | 0.92 | 2.98 | 3.24) | 3.33 ± 0.07 | 21 |
| | 0.78 | 2.67 | 3.42) | | |

Injection of 1 unit of cytostatic factor 2 hours, 1 day or 2 days before injection of 125 units of IL-1 still caused significant inhibition. The inhibition was lower if the time between was increased to 3 days and only marginal by 4 days. A significant inhibition was also observed if the cytostatic factor was injected 2 hours after the IL-1, but this decreased when the interval was increased to 1 day.

### Alternative Purification and Activity

Tissue Culture

The M20 cells were grown as previously described (Barak et al., 1986). Briefly, the cells were grown at 37° C. and 5% CO₂ in RPMI 1640 supplemented with penicillin (50 U/mJ), streptomycin (5 μg/ml), MEM vitamin solution (1:100), 2 mM L-glutamine, 1 mM sodium pyruvate (complete RMPI) with the addition of 5% FCS (Beit Haemek, Israel) for 4 days followed by a 24 hour incubation at 10⁶ cells/ml in complete RPMI without FCS. The conditioned medium was collected and concentrated to 10 Ml by vacuum ultrafiltration using dialysis tubing (cut-off 10,000 MW).

Bioassay

The assay used was a modified LAF assay as described by Gery et al. (Gery et al., 1972). Briefly, thymocyte from one month old C3H/HEJ mice were made into single cell suspension in complete RPMI. 2-mercaptoethanol (50 μM), PHA (1 μg/ml) and 4-6 units/ml of rIL-1B were added to each well to establish a positive growth. The cells were plated at 5×10⁶ thymocytes/well in 100 μl. The fractions to be tested for inhibitory activity were added at 25 μl/well. The cultures were grown at 37° C. in 5% CO₂ for 72 hours with the addition of 1 μCi/well of ³H-thymidine (specific activity 6.7 Ci/mmol) for the last 12 hours, harvested and DNA synthesis was measured using standard techniques.

Gel Filtration

A concentrated sample from the M20-2 supernatant was dialyzed against the eluant buffer (PBS+0.5M NaCl) and applied to a Sephacryl S-300 open column (800 ml Vₜ and 230 ml Vₒ). The column was run using a peristaltic pump at a flow rate of 90 ml/hour. After the volume corresponding to Vₒ has passed through the column, fractions of 7 ml were collected, and absorbance at 280 nm was determined. One peak showing inhibitory activity in the bioassay, was pooled and concentrated by vacuum ultrafiltration as described above, and dialyzed against distilled water, overnight.

Affi-Gel Blue Chromatography

To remove the bulk of the BSA, which is a major protein in the tissue culture medium, 2 liters of culture medium were concentrated by vacuum ultrafiltration and passed through an Affi-Gel blue column (BioRad) of 11.0×2.6 cm (bed volume 58 ml), previously equilibrated with 50 mM Tris buffer pH 7.5 containing 50 mM NaCl. The effluent from the column was applied to the Rotofor cell as described.

Preparative Isoelectric Focusing Isoelectric focusing (IEF) was performed using a Rotofor cell (BioRad, Richmond, Calif.). The concentrated sample obtained following gel filtration or Affi-Gel blue chromatography was mixed with 10M urea to obtain a final concentration of 5M. Carrier ampholytes (Bio-lyte), covering the pH range of 3-5 were added to obtain a final concentration of 10%. The volume of the mixture was brought to 50 ml with distilled water. To bring the sample to the running temperature (4° C.), the cell was rotated for 15 minutes before applying the electric current. The run was carried out at constant power (12 W). In a typical run, the initial conditions were 784 V and 15 mA. At equilibrium, after 4 hours, when the run was stopped, the voltage reached 1170 V and the current was 10 mA. At the completion of the run the pH gradient was determined on the 20 harvested fractions, on aliquots diluted 1:10 in freshly boiled twice distilled water. To remove the carrier ampholytes before the bioassay the harvested fractions, to which 0.1 mg/ml crystalline BSA was added, were dialyzed against 1M NaCl for 24 hours followed by dialysis against RPMI 1640 medium for 48 hours.

To increase the degree of purification obtained by IEF in the Rotofor cell, a second run in a narrower pH range corresponding to the fractions in which the cytostatic factor has focused, was attempted. The harvested fractions obtained in tie first run (pIs 3.9-4.3) were pooled, diluted with glycerol (10% final concentration) and twice distilled water, and reloaded into the Rotofor cell. The run was performed as described above. In a typical run, the starting power conditions were: constant power 12W; 1087 V and 12 mA. At the end of the run, the voltage reached 1828 V and current dropped to 7 mA. The harvested fractions were treated as described above for the fractions obtained, when only one run in the Rotofor cell was performed.

Protein Concentration

The determination of the protein concentration of the samples was performed using the Bradford microassay procedure with the Bio-Rad protein assay kit. To avoid the interference of carrier ampholytes, the samples were acidified with HCl, before adding the Coomassie Brilliant Blue G-250 reagent, as described by Ramagli and Rodriguez in Electrophoresis 6, 559 (1985). Absorbance at 595 nm was measured against a reagent blank using the GIlford 240 spectrophotometer. Crystalline BSA was used as standard.

Polyacrylamide Gel Isoelectric Focusing PAGIF)

Analytical isoelectric focusing was performed in a fabric reinforced polyacrylamide gel (5% T and 3% C) of 150 μm thickness, containing 5% carrier ampholytes (Servalyt), pH range 3-10. The run was performed at 4-9° C. for about 3 hours at 3W constant power. Ten μl of the samples were applied in slots of 2×3.5 mm in the middle of the gel. As anolyte a mixture of 25 mM L-aspartic acid and 25 mM L-glutamic acid was used. A mixture composed of 23 mM L-arginine, 27 mM L-lysine and 1.78 M ethylene diamine served as catholyte. When the run was performed with carrier ampholytes pH 3-6, the 10 μl samples were applied in slots of 7×1 mm in the middle of the gel. The anolyte was the same mixture as described above, while as catholyte a 0.27 M glycine was used. The low pI calibration kit (pH 2.5-6.5) (Pharmacia, Uppsala, Sweden) was the source of the pI markets.

SDS-PAGE

Samples, heated at 95° C. for 4 minutes is sample buffer, were run in a 10-15% gradient SDS-PAGE using the Bio-Rad Mini Protean II cell. The gel was run for 1 hour at 200V. The gel was stained with Coomassie brilliant blue R-250 (Bio-Rad).

FPLC Gel Filtration

To fractionate the mixture of proteins according to their differences in molecular dimensions, gel filtration through a Superose 12/10 column (Pharmacia, LKB Biotechnology, Uppsala, Sweden) was used. The sample was injected into a 0.5 ml loop and the column was run at a flow rate of 0.25 ml/min with PBS, for 40 minutes. The samples were prepared for the bioassay by addition of 0.1 mg/ml BSA as a stabilizer and dialyzed against RPM1 1640 for 48 hours. The column was calibrated using human hemoglobin (Sigma, St. Louis Mo.) and BioRad Molecular weight marker kit (bovine thyroglobulin, bovine γ-globulin, chicken ovalbumin, Horse myoglobin, and vitamin B12).

TABLE V
Purification of the M20 cytostatic factor from
conditioned medium

| | Specific | Purification | |
|---|---|---|---|
| Step | activity* (U/μg) | fold | Yield (%) |
| Conditioned | O.015** | 1 | 100.0 |
| medium | | | |
| Sephacryl S-300 | 1.060 | 71 | 20.0 |
| IEF pH 3-5 | 15.900 | 1060 | 7.6 |
| FPLC Superose 12 | 24.200 | 1613 | 5.4 |

*One unit of cytostatic factor is defined as the amount of material (μg) causing 50% inhibition of the proliferation in the thymocyte bioassay, induced by one unit of RIL-1B.
**Since IL-1 which is concomitantly present in the crude conditioned medium interferes with the measurement of the cytostatic factor used in the bioassay, its specific activity is underestimated.

Deposit of Microoganisms

The following M20-2 cell line has been deposited on Apr. 4, 1990 with the American Type Culture Collection, Rockville, Md., and has been assigned the listed accession number:

$$\frac{Cell Line}{M20 - 2} \frac{Accession Number}{ATCC CRL 10409}$$

The present invention is not to be limited in scope by the cell line deposited since the deposited embodiment is intended as a single illustration of individual aspects of the invention, and any cell lines which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims.

What is claimed is:

1. A purified and isolated 52±8 kD molecular weight protein, free of the cell line M20 and M20-2, said protein being characterized by a pI of 4.1 and by having general cytostatic but not cytotoxic activity.

2. A purified and isolated 52±4 kD molecular weight protein, free of the cell line M20 and M20-2, said protein being characterized by a pI of 4.1 and by having general cytostatic but not cytotoxic activity.

3. A protein of claim 1 or claim 2, wherein the cytostatic activity is on immune cells.

4. A purified and isolated 52±8 kD molecular weight protein, free of the cell line M20-2, said protein being characterized by a pI of 4.1 and the inhibition or reduction of an inflammatory response.

5. A purified and isolated 52±4 kD molecular weight protein, free of the cell line M20-2, said protein being characterized by a pI of 4.1 and the inhibition or reduction of an inflammatory response.

6. A purified and isolated 52±8 kD molecular weight protein, free of the cell line M20-2, said protein being characterized by a pI of 4.1 and the inhibition or reduction of IL-1 associated effects.

7. A purified and isolated 52±4 kD molecular weight protein, free of the cell line M20-2, said protein being characterized by a pI of 4.1 and the inhibition or reduction of IL-1 associated effects.

8. A protein of claim 6 or claim 7, wherein one of the IL-1 associated effects is an inflammatory response.

9. A therapeutic composition comprising an anti-inflammatory-effective amount of the protein of claim 1 in combination with a pharmaceutically acceptable carrier.

10. A method of inhibition or reduction of an inflammatory response which comprises administering to an individual in need of such treatment an effective amount of the protein of claim 1.

11. The method of claim 10 wherein the amount is about 0.0001-1.0 μg per kg of body weight.

12. A substantially pure protein free of major contaminants as shown by the presence of one band in SDS-PAGE, the protein having general cytostatic but not cytotoxic activity and the same amino acid sequence and isoelectric point as the cytostatic factor protein with an isoelectric point of 4.1±0.2 obtained from M20-2 cells, and active fragments thereof.

13. A protein of claim 12, wherein the molecular weight is 54±8.

14. A protein of claim 12, wherein the molecular weight is 54±4.
